(19) United States
(12) Patent Application Publication
(10) Pub. No.: US 2016/0031128 A1
(43) Pub. Date: Feb. 4, 2016

(54) METHOD FOR MANUFACTURING ACOUSTIC COUPLING MEMBER

(71) Applicant: SEIKO EPSON CORPORATION, Tokyo (JP)

(72) Inventor: Kanechika KIYOSE, Matsumoto (JP)

(21) Appl. No.: 14/805,769

(22) Filed: Jul. 22, 2015

(30) Foreign Application Priority Data
Jul. 31, 2014 (JP) 2014-156709

(51) Int. Cl. B29C 43/14; A61B 8/08; A61B 8/00

(52) U.S. Cl. CPC B29C 43/146; A61B 8/4281; A61B 8/461; A61B 8/4455; A61B 8/5207; B29K 2083/00

(57) ABSTRACT

A first mold has a recessed surface portion. A second mold that has a protruding surface portion and a flat surface portion is laid on top of the first mold, thereby forming a first cavity between the recessed surface portion and the protruding surface portion and a second cavity between the first mold and the flat surface portion. The first cavity and the second cavity are filled with a first resin material. A third mold having a flat inner wall surface is laid on top of the first mold and the first resin, thereby forming a space between the inner wall surface and the first resin so as to correspond to the protruding surface portion. This space is filled with a second resin material, and thus an acoustic coupling member is molded.

### BACKGROUND

1. Technical Field

The present invention relates to a method for manufacturing an acoustic coupling member, and the like.

2. Related Art

As described in JP-A-2010-213983, an ultrasonic probe includes an acoustic lens, which is a specific example of an acoustic coupling member. The acoustic lens is formed to have a two-layer structure. In a first member, a flat surface coupled to an ultrasonic vibrator and an outwardly-curved partial cylindrical surface having generating lines that are parallel to the flat surface are defined. A second member is coupled to the partial cylindrical surface, the second member being curved along the partial cylindrical surface and forming an outer covering of the partial cylindrical surface.

The second member is bonded to the partial cylindrical surface of the first member with an adhesive. A depression having a partial cylindrical surface that conforms to the partial cylindrical surface of the first member is formed in the second member. The adhesive is applied to the partial cylindrical surface of the first member or the depression. After removal of bubbles, the two members are bonded together. Since the adhesive is a fluid, it is difficult to sandwich the adhesive between the partial cylindrical surface of the first member and the depression such that the adhesive has a uniform film thickness. The variation in film thickness of the adhesive affects the acoustic characteristics of the acoustic coupling member. Here, in, for example, Japanese Patent No. 4873673, a urethane resin coating layer is applied to a surface of a base to realize a two-layer structure. However, the urethane resin coating layer is a coating film and cannot have a thick portion that extends outward from an outline of a thin portion continuously therewith and that has a larger thickness than the thickness of the thin portion.

### SUMMARY

According to at least one aspect of the invention, it is possible to provide a method for manufacturing an acoustic coupling member that is a coupled body of two members but is not affected by the film thickness of an adhesive.

(1) An aspect of the invention is directed to a method for manufacturing an acoustic coupling member, the method including laying, on top of a first mold that has a recessed surface portion, a second mold that has a protruding surface portion having a certain height from a reference plane and a flat surface portion extending in the reference plane continuously with the protruding surface portion, thereby forming a first cavity between the recessed surface portion and the protruding surface portion and a second cavity between the first mold and the flat surface portion, where the second cavity is continuous with the first cavity and has a larger cavity thickness than a cavity thickness of the first cavity, and filling the first cavity and the second cavity with a first resin material; hardening the first resin material, thereby molding a resin body having a thin portion located within the first cavity and a thick portion located within the second cavity, the thick portion extending outward from an outline of the thin portion continuously therewith and having a larger thickness than a thickness of the thin portion; laying a third mold, the third mold having a flat inner wall surface that comes into contact with the thick portion of the resin body, on top of the first mold, thereby forming a space between the inner wall surface and a depression that is formed in the resin body so as to correspond to the protruding surface portion, and filling the space with a second resin material; and hardening the second resin material such that the second resin material is continuous with the resin body, thereby molding an acoustic coupling member.

The second resin material is hardened such that it is continuous with the resin body formed of the first resin material. As a result, the second resin material can be cured into a shape that conforms to the depression of the resin body. At the same time as the hardening of the second resin material, a cured product of the second resin material is coupled to the resin body of the first resin material. Therefore, formation of an adhesive layer between the resin body and the cured product can be avoided. In this manner, even though the acoustic coupling member is a coupled body of the two members, the acoustic coupling member can be free from the influence of an adhesive. A method for manufacturing an acoustic coupling member that is not affected by the film thickness of an adhesive is thus provided.

(2) It is also possible that after the first resin material is cured, the second mold is detached from the first mold, and the third mold is laid on top of the first mold with the resin body remaining on the first mold. When the second mold is detached from the first mold, and the third mold is laid on top of the first mold, the molded resin body is held on the first mold without being released from the first mold. During curing of the second resin material, the first mold is used as is, so that mold-use efficiency is increased. The manufacturing cost is thus suppressed.

(3) Alternatively, it is also possible that after the first resin material is cured, the resin body is released from the first mold, and the resin body is transferred onto a mold having a cavity surface that has the same shape as the first mold. The molded resin body is released from the first mold. The resin body is transferred from the first mold onto the next mold. After the transfer, the third mold is laid on top of that mold. The time and effort taken to transport the first mold can thus be eliminated.

(4) It is also possible that flash is formed between the first mold and the second mold, extending outward from an outline of the resin body. The flash can be used during mold release. The resin body can be easily pulled out of the first mold. After the mold release, the flash can be removed.

(5) The method for manufacturing an acoustic coupling member may further include applying a third resin material to a surface of the resin body, the surface conforming to the first mold. A cured product of the third resin material protects the surface of the resin body.

(6) It is also possible that the protruding surface portion constitutes a partial cylindrical surface having generating lines that are parallel to the reference plane, and the protruding surface portion is positioned facing the recessed surface portion that is defined in the first mold, the recessed surface portion constituting a depression that constitutes a partial cylindrical surface having generating lines that are parallel to said generating lines, and demarcates the first cavity between the protruding surface portion and the first mold. The acoustic coupling member functions as an acoustic lens.

(7) It is also possible that the thick portion is formed into a frame shape that surrounds a perimeter of the thin portion. The thick portion is less likely to be broken than the thin portion. Since the edge of the thin portion is continuously surrounded by the thick portion, breakage of the thin portion can be sufficiently avoided during, for example, mold release.

(8) Another aspect of the invention is directed to an acoustic coupling member including a thin portion that is formed of a first resin material and that extends, at a position separated from a reference plane, along the reference plane, a thick portion that is formed of the first resin material, that has a larger thickness than a thickness of the thin portion, and that defines a flat surface, the flat surface extending outward from an outline of the thin portion continuously therewith and being in contact with the reference plane, and a connecting portion that is formed of a second resin material, the second resin material being different from the first resin material, and that fills a space between the reference plane and the thin portion and is in close contact with the thin portion.

The connecting portion formed of the second resin material is directly coupled to the thin portion formed of the first resin material. Therefore, formation of an adhesive layer between the thin portion and the connecting portion can be avoided. Even though the acoustic coupling member is a coupled body of the first resin material and the second resin material, the acoustic coupling member can be free from the influence of an adhesive. A method for manufacturing an acoustic coupling member that is not affected by an adhesive is thus provided.

(9) It is preferable that the second resin material has a Shore hardness value that is smaller than that of the first resin material. The acoustic coupling member comes into contact with an object at its thin portion. Ultrasonic waves propagate through the thin portion and the connecting portion. Since the thin portion has a Shore hardness value that is larger than that of the connecting portion, the thin portion can exhibit higher resistance to wear than the connecting portion.

(10) It is also possible that the second resin material contains filler, a value of an amount of said filler that is contained being smaller than that of the first resin material. The acoustic impedance of the second resin material can be reduced according to the amount of filler that is reduced. Therefore, the overall acoustic impedance of the acoustic coupling member is reduced when compared with the case where the acoustic coupling member is entirely formed of the first resin material. Ultrasonic waves propagating through the acoustic coupling member are increased. The sensitivity with respect to ultrasonic waves can thus be enhanced.

(11) It is also possible that the thick portion is formed into a frame shape that surrounds a perimeter of the thin portion. The thick portion is less likely to be broken than the thin portion. Since the edge of the thin portion is continuously surrounded by the thick portion, breakage of the thin portion can be sufficiently avoided during, for example, mold release.

(12) It is also possible that a surface of the thin portion, the surface being located on a side opposite to the reference plane, is formed by a partial cylindrical surface having generating lines that are parallel to the reference plane. The acoustic coupling member functions as an acoustic lens.

(13) The acoustic coupling member can be used in a state in which it is incorporated into a probe. At this time, it is sufficient if the probe includes the acoustic coupling member, an ultrasonic device that is coupled to the acoustic coupling member, and a housing that supports the acoustic coupling member.

(14) The acoustic coupling member can be used in a state in which it is incorporated into an electronic apparatus. At this time, it is sufficient if the electronic apparatus includes the acoustic coupling member, an ultrasonic device that is coupled to the acoustic coupling member, and a processing unit that is connected to the ultrasonic device and processes an output from the ultrasonic device.

(15) The acoustic coupling member can be used in a state in which it is incorporated into an ultrasonic imaging apparatus. At this time, it is sufficient if the ultrasonic imaging apparatus includes the acoustic coupling member, an ultrasonic device that is coupled to the acoustic coupling member, a processing unit that is connected to the ultrasonic device, processes an output from the ultrasonic device, and generates an image, and a display device that displays the image.

### BRIEF DESCRIPTION OF THE DRAWINGS

The invention will be described with reference to the accompanying drawings, wherein like numbers reference like elements.

FIG. 1 is an external view schematically showing a specific example, that is, an ultrasonic diagnostic apparatus, of an electronic apparatus according to an embodiment of the invention.

FIG. 2 is an enlarged plan view of an ultrasonic device according to the embodiment.

FIG. 3 is a partial cross-sectional view of the ultrasonic device according to the embodiment taken along line A-A in FIG. 1.

FIG. 4 is an enlarged cross-sectional perspective view of an acoustic lens.

FIG. 5 is a perspective view schematically showing the shape of a first mold.

FIG. 6 is a perspective view schematically showing the shape of a second mold.

FIG. 7 is a cross-sectional view schematically showing the second mold that is laid on top of the first mold.

FIG. 8 is a cross-sectional view schematically showing an outer covering that is held on the first mold after curing of a first resin material.

FIG. 9 is a cross-sectional view schematically showing a third mold that is laid on top of the first mold.

FIG. 10 is a cross-sectional view schematically showing the outer covering that is temporarily released from the first mold after curing of the first resin material.

FIG. 11 is an enlarged cross-sectional perspective view corresponding to FIG. 4 and showing an acoustic lens according to another embodiment.

FIG. 12 is a partial cross-sectional view corresponding to FIG. 3 and showing an ultrasonic device according to another embodiment.

### DESCRIPTION OF EXEMPLARY EMBODIMENTS

The following describes embodiments of the invention with reference to the attached drawings. It should be noted that the embodiments to be described hereinafter are not intended to unduly limit the scope of the invention defined by the claims and that not all of the configurations to be described in the embodiments are necessarily essential as the means for achieving the invention.

(1) Overall Configuration of Ultrasonic Diagnostic Apparatus

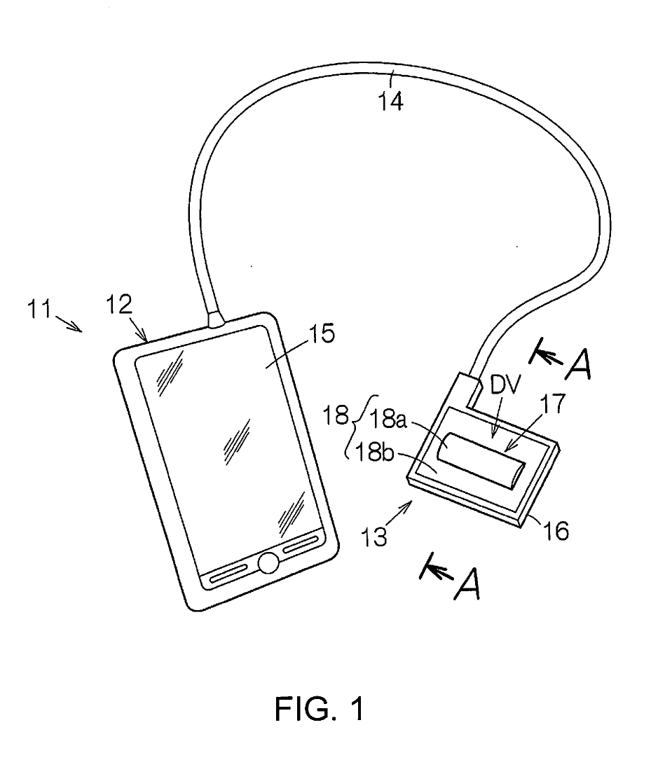

FIG. 1 schematically shows the configuration of a specific example, that is, an ultrasonic diagnostic apparatus (ultrasonic imaging apparatus) 11, of an electronic apparatus according to an embodiment of the invention. The ultrasonic diagnostic apparatus 11 includes a device terminal (processing unit) 12 and an ultrasonic probe (probe) 13. The device terminal 12 and the ultrasonic probe 13 are connected to each other via a cable 14. Electric signals are transmitted through the cable 14 between the device terminal 12 and the ultrasonic probe 13. A display panel (display device) 15 is incorporated into the device terminal 12. A screen of the display panel 15 is exposed at a surface of the device terminal 12. In the device terminal 12, an image is generated based on ultrasonic waves detected by the ultrasonic probe 13. The imaged detection result is displayed on the screen of the display panel 15.

The ultrasonic probe 13 has a housing 16. An ultrasonic device unit DV is fitted in the housing 16. The ultrasonic device unit DV includes an ultrasonic device 17. The ultrasonic device 17 includes an acoustic lens (acoustic coupling member) 18. A partial cylindrical surface 18a is formed on an outer surface of the acoustic lens 18. The partial cylindrical surface 18a is surrounded by a flat plate portion 18b. The entire outer perimeter of the flat plate portion 18b is continuously joined to the housing 16. Thus, the flat plate portion 18b functions as a portion of the housing. The acoustic lens 18 may be formed of, for example, a silicone resin. The acoustic lens 18 has an acoustic impedance that is similar to the acoustic impedance of a living body. The ultrasonic device 17 outputs ultrasonic waves from its surface and receives reflected waves of the ultrasonic waves.

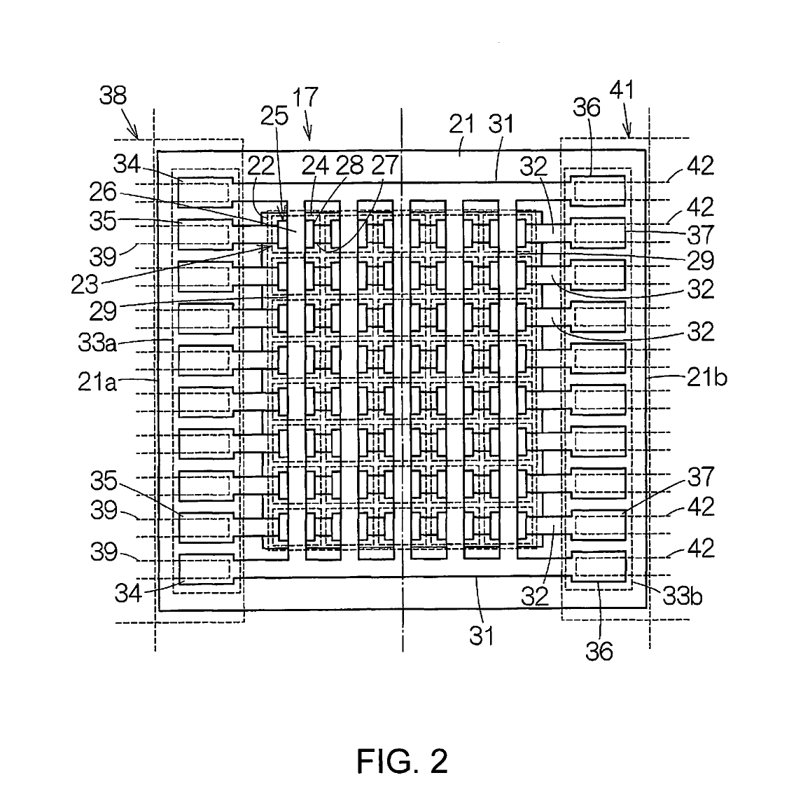

FIG. 2 schematically shows a plan view of the ultrasonic device 17. The ultrasonic device 17 includes a base 21. An element array 22 is formed on a surface (first surface) of the base 21. The element array 22 is constituted by an arrangement of thin-film ultrasonic transducer elements (hereinafter referred to as “elements”) 23 that are arranged in an array. The arrangement is in the form of a matrix having a plurality of columns and a plurality of rows. The arrangement may also be established as a staggered arrangement. In a staggered arrangement, a group of elements 23 in each even row can be displaced relative to a group of elements 23 in each odd row by one-half of the column pitch. Either the number of elements in each odd row or the number of elements in each even row may be smaller than the other by one.

Each element 23 includes a vibration film 24. In FIG. 2, the outline of the vibration film 24 when viewed from above in a direction perpendicular to the film surface of the vibration film 24 (when viewed from above in a thickness direction of a substrate) is shown by dashed lines. A piezoelectric element 25 is formed on the vibration film 24. The piezoelectric element 25 is constituted by a top electrode 26, a bottom electrode 27, and a piezoelectric film 28. In each element 23, the piezoelectric film 28 is sandwiched between the top electrode 26 and the bottom electrode 27. The bottom electrode 27, the piezoelectric film 28, and the top electrode 26 are laid one on top of another in that order. The ultrasonic device 17 is configured as a single ultrasonic transducer element chip (substrate).

A plurality of first electric conductors 29 are formed on the surface of the base 21. The first electric conductors 29 extend parallel to one another in a column direction of the arrangement. One first electric conductor 29 is assigned to corresponding one column of elements 23. One first electric conductor 29 is connected commonly to the piezoelectric films 28 of the respective elements 23 that are lined up in the column direction of the arrangement. The first electric conductor 29 forms the top electrodes 26 for the respective elements 23. Both ends of the first electric conductor 29 are respectively connected to a pair of extraction interconnects 31. The extraction interconnects 31 extend parallel to each other in a row direction of the arrangement. Accordingly, all of the first electric conductors 29 have the same length. Thus, the top electrodes 26 are connected commonly to the elements 23 of the entire matrix. The first electric conductors 29 can be formed of, for example, iridium (Ir). However, other electrically conductive materials may also be used for the first electric conductors 29.

A plurality of second electric conductors 32 are formed on the surface of the base 21. The second electric conductors 32 extend parallel to one another in the row direction of the arrangement. One second electric conductor 32 is assigned to corresponding one row of elements 23. One second electric conductor 32 is connected commonly to the piezoelectric films 28 of the respective elements 23 that are lined up in the row direction of the arrangement. The second electric conductor 32 forms the bottom electrodes 27 for the respective elements 23. For example, a laminated film composed of titanium (Ti), iridium (Ir), platinum (Pt), and titanium (Ti) can be used for the second electric conductors 32. However, other electrically conductive materials may also be used for the second electric conductors 32.

Energization of the elements 23 is switched on a row-by-row basis. A linear scan and a sector scan can be achieved in accordance with this switching of energization. Since the elements 23 in a single row simultaneously output ultrasonic waves, the number of elements in a single row, that is, the number of columns of the arrangement can be determined in accordance with the output level of ultrasonic waves. The number of columns can be set at, for example, about 10 to 15. In FIG. 2, some columns are not shown, and only five columns are shown. The number of rows of the arrangement can be determined in accordance with the extent of the scan range. The number of rows can be set at, for example, 128 or 256. In FIG. 2, some rows are not shown, and only eight rows are shown. The functions of the top electrodes 26 and the bottom electrodes 27 may be reversed. That is to say, it is also possible that while the bottom electrodes are connected commonly to the elements 23 of the entire matrix, the top electrodes are connected commonly to the elements 23 in each row of the arrangement.

The outline of the base 21 has a first side 21a and a second side 21b that are defined by a pair of mutually parallel straight lines and that oppose each other. A first terminal array 33a in a single line is disposed between the first side 21a and the outline of the element array 22. A second terminal array 33b in a single line is disposed between the second side 21b and the outline of the element array 22. The first terminal array 33a can form a single line parallel to the first side 21a. The second terminal array 33b can form a single line parallel to the second side 21b. The first terminal array 33a is constituted by a pair of top electrode terminals 34 and a plurality of bottom electrode terminals 35. Similarly, the second terminal array 33b is constituted by a pair of top electrode terminals 36 and a plurality of bottom electrode terminals 37. One top electrode terminal 34 and one top electrode terminal 36 are respectively connected to the two ends of a single extraction interconnect 31. It is sufficient if the extraction interconnects 31 and the top electrode terminals 34 and 36 are formed plane-symmetrically with respect to a perpendicular plane that bisects the element array 22. One bottom electrode terminal 35 and one bottom electrode terminal 37 are respectively connected to the two ends of a single second electric conductor 32. It is sufficient if the second electric conductors 32 and the bottom electrode terminals 35 and 37 are formed plane-symmetrically with respect to the perpendicular plane that bisects the element array 22. Here, the base 21 is formed to have a rectangular outline. The outline of the base 21 may also be square or may be trapezoidal.

A first flexible printed wiring board (hereinafter referred to as “first wiring board”) 38 is connected to the base 21. The first wiring board 38 covers the first terminal array 33a. Electrically conductive lines, namely, first signal lines 39 are formed at one end of the first wiring board 38, respectively corresponding to the top electrode terminals 34 and the bottom electrode terminals 35. The first signal lines 39 are respectively opposed to the top electrode terminals 34 and the bottom electrode terminals 35 and respectively joined thereto. Similarly, a second flexible printed wiring board (hereinafter referred to as “second wiring board”) 41 covers the base 21. The second wiring board 41 covers the second terminal array 33b. Electrically conductive lines, namely, second signal lines 42 are formed at one end of the second wiring board 41, respectively corresponding to the top electrode terminals 36 and the bottom electrode terminals 37. The second signal lines 42 are respectively opposed to the top electrode terminals 36 and the bottom electrode terminals 37 and respectively joined thereto.

(2) Configuration of Ultrasonic Device

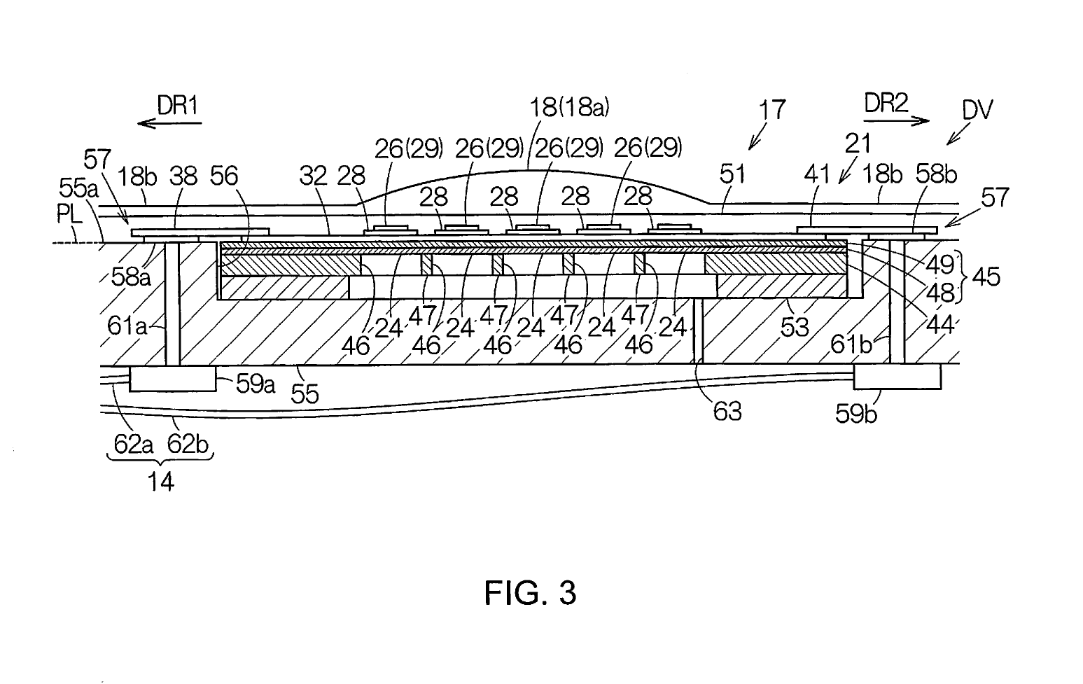

As shown in FIG. 3, the base 21 includes a substrate 44 and a coating film 45. The coating film 45 is formed over the entire surface of the substrate 44. In the substrate 44, an opening 46 is formed for each of the elements 23. The openings 46 are arranged in an array in the substrate 44. The openings 46 for the respective elements 23 open in a surface (second surface) on the back side (opposite side). The outline of a region where the openings 46 are arranged corresponds to the outline of the element array 22. A partitioning wall 47 is disposed between every two adjacent openings 46. Adjacent openings 46 are separated from each other by the partitioning walls 47. The wall thickness of the partitioning walls 47 corresponds to the spacing between the openings 46. Each partitioning wall 47 defines two wall surfaces within planes that extend parallel to each other. The wall thickness corresponds to the distance between the two wall surfaces. That is to say, the wall thickness can be defined by the length of a normal line that extends between the wall surfaces orthogonally to the wall surfaces. The substrate 44 can be formed of, for example, a silicon substrate.

The coating film 45 is composed of a silicon oxide (SiO₂) layer 48 that is laminated on the surface of the substrate 44 and a zirconium oxide (ZrO₂) layer 49 that is laminated on a surface of the silicon oxide layer 48. The coating film 45 closes the spaces in the openings 46. Thus, portions of the coating film 45 that correspond to the respective outlines of the openings 46 form the vibration films 24. The vibration films 24 refer to those portions of the coating film 45 that face the respective openings 46 and that can thus vibrate in the thickness direction of the substrate 44. The film thickness of the silicon oxide layer 48 can be determined based on resonance frequency.

The bottom electrode 27, the piezoelectric film 28, and the top electrode 26 are sequentially laminated on the surface of each vibration film 24. The piezoelectric film 28 can be formed of, for example, lead zirconate titanate (PZT). Other piezoelectric materials may also be used for the piezoelectric film 28. Here, the piezoelectric film 28 under the first electric conductor 29 completely covers the second electric conductor 32. The piezoelectric film 28 can serve to avoid short-circuiting between the first electric conductor 29 and the second electric conductor 32.

The acoustic matching layer 51 is laminated over the surface of the base 21. The acoustic matching layer 51 covers the element array 22. The film thickness of the acoustic matching layer 51 is determined in accordance with the resonance frequency of the vibration films 24. For example, a silicone resin film can be used for the acoustic matching layer 51. The acoustic lens 18 is disposed on the acoustic matching layer 51. A flat surface of the acoustic lens 18 on the back side of the partial cylindrical surface 18a is in close contact with a surface of the acoustic matching layer 51. The acoustic matching layer 51 serves to allow the acoustic lens 18 to adhere to the base 21. Generating lines of the partial cylindrical surface 18a are positioned parallel to the first electric conductors 29. The curvature of the partial cylindrical surface 18a is determined in accordance with the focus position of ultrasonic waves emitted from a single row of elements 23 connected to a single second electric conductor 32.

A reinforcing plate 53 serving as a backing material is coupled to the back surface of the base 21. The reinforcing plate 53 is formed into a flat plate shape. The back surface of the base 21 is superposed on a surface of the reinforcing plate 53. The surface of the reinforcing plate 53 is joined to the back surface of the base 21. To join the reinforcing plate 53 to the base 21 as described above, the reinforcing plate 53 may be bonded to the base 21 with an adhesive. The reinforcing plate 53 reinforces the stiffness of the base 21. The reinforcing plate 53 serves to secure a favorable flatness of the surface of the base 21. The reinforcing plate 53 can be provided with a rigid base material, for example. This base material can be formed of a metal material such as 42 Alloy (iron-nickel alloy), for example.

The ultrasonic device unit DV includes a wiring substrate 55. The wiring substrate 55 is coupled to the ultrasonic device 17. The wiring substrate 55 has a flat surface portion 55a that extends in a plane PL and a recess 56 that is recessed from the flat surface portion 55a. The recess 56 defines the shape of the outline of the base 21 when viewed from above. The ultrasonic device 17 is received in the recess 56. Here, the surface of the base 21 of the ultrasonic device 17 is contained in the plane PL of the wiring substrate 55. Thus, the ultrasonic device 17 is positioned flush with the plane PL. The ultrasonic device 17 may be fixed to the wiring substrate 55 with a resin material.

A wiring pattern 57 is formed on the wiring substrate 55. The first wiring board 38 and the second wiring board 41 of the ultrasonic device 17 are connected to the wiring pattern 57. The wiring pattern 57 includes first electrically conductive pads 58a and second electrically conductive pads 58b. The first electrically conductive pads 58a and the second electrically conductive pads 58b are formed on the plane PL of the wiring substrate 55. The individual first electrically conductive pads 58a and second electrically conductive pads 58b are arranged so as to correspond to the respective first signal lines 39 and second signal lines 42. The first electrically conductive pads 58a and the second electrically conductive pads 58b can be formed of an electrically conductive material such as copper, for example. The first signal lines 39 and the second signal lines 42 are respectively joined to the corresponding first electrically conductive pads 58a and second electrically conductive pads 58b.

One end of the first wiring board 38 is laid on top of and connected to the ultrasonic device 17 at a position higher than the plane PL of the wiring substrate 55. The first wiring board 38 extends in a first direction DR1 from the one end that is located on the ultrasonic device 17. The other end of the first wiring board 38 is laid on top of and connected to the plane PL of the wiring substrate 55. The first wiring board 38 overlaps the plane PL while being spaced apart therefrom by a distance corresponding to the thickness of the first electrically conductive pads 58a. Similarly, one end of the second wiring board 41 is laid on top of and connected to the ultrasonic device 17 at a position higher than the plane PL of the wiring substrate 55. The second wiring board 41 extends in a second direction DR2 from the one end that is located on the ultrasonic device 17. The second direction DR2 is opposite to the first direction DR1. The other end of the second wiring board 41 is laid on top of and connected to the plane PL of the wiring substrate 55. The second wiring board 41 overlaps the plane PL while being spaced apart therefrom by a distance corresponding to the thickness of the second electrically conductive pads 58b.

First connectors 59a and second connectors 59b are arranged on a back surface of the wiring substrate 55. Each first connector 59a is connected to the corresponding first electrically conductive pad 58a through a via hole 61a. Each second connector 59b is connected to the corresponding second electrically conductive pad 58b through a via hole 61b. The via holes 61a and 61b pass through the wiring substrate 55 from the surface to the back surface thereof. The cable 14 is formed by wires 62a and 62b that are respectively connected to the first connectors 59a and the second connectors 59b.

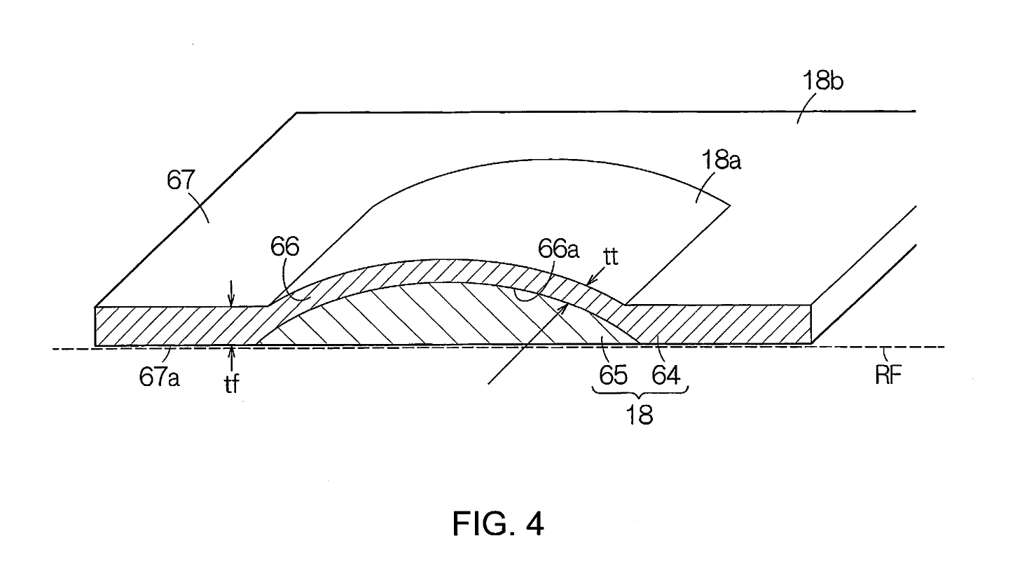

As shown in FIG. 4, the acoustic lens 18 is formed to have a two-layer structure. The acoustic lens 18 having the two-layer structure has an outer covering 64 and a connecting body (connecting portion) 65. The outer covering 64 is formed of a first resin material. For example, a millable silicone rubber may be used as the first resin material. Here, the millable silicone rubber can contain, for example, a silicone rubber having a dimethylpolysiloxane structure containing a vinyl group, silica, and a vulcanizing agent. Specifically, it is sufficient if silica is mixed as silica particles having a weight-average particle diameter of 15 μm to 30 μm at a mass ratio between 40 mass % and 50 mass % inclusive with respect to the silicone rubber. For example, 2,5-dimethyl-2,5-ditertiary butyl peroxyhexane can be used as the vulcanizing agent. The connecting body 65 is formed of a second resin material. The second resin material differs from the first resin material. For example, an RTV silicone rubber containing no filler such as silica may be used as the second resin material. Here, mixing of a resin agent is avoided as far as possible. A cured product of the second resin material exhibits lower attenuation of ultrasonic waves than a cured product of the first resin material. The cured product of the second resin material has a Shore hardness value that is smaller than that of the cured product of the first resin material.

The outer covering 64 is formed by a thin portion 66 and a thick portion 67. The thin portion 66 extends, at a position separated from a reference plane RF, along the reference plane RF. An outer surface of the thin portion 66 defines the partial cylindrical surface 18a. The partial cylindrical surface 18a has generating lines that are parallel to the reference plane RF. Similarly, an inner surface 66a of the thin portion 66 forms a partial cylindrical surface. The inner surface 66a has generating lines that are parallel to the generating lines of the partial cylindrical surface 18a. The thin portion 66 covers the region of the element array 22. The thickness tt of the thin portion 66 can be derived from, for example, a difference in radius of curvature between the outer surface (18a) and the inner surface 66a.

The thick portion 67 extends outward from the outline of the thin portion 66 continuously therewith. The thick portion 67 is disposed outside the region of the element array 22. Outside the region of the element array 22, the thick portion 67 defines a flat surface 67a that is in contact with the reference plane RF. The thick portion 67 has the thickness tf that is larger than the thickness tt of the thin portion 66. The thickness tf of the thick portion 67 can be measured as a thickness from the reference plane RF. Here, the thick portion 67 is formed into a frame shape that surrounds the perimeter of the thin portion 66. The thick portion 67 forms the above-described flat plate portion 18b. A space is demarcated between the inner surface 66a of the thin portion 66 and the reference plane RF.

The connecting body 65 fills the space between the inner surface 66a of the thin portion 66 and the reference plane RF. The connecting body 65 is in close contact with the inner surface 66a of the thin portion 66. Contamination with air bubbles is thus eliminated between the connecting body 65 and the thin portion 66.

(3) Operation of Ultrasonic Diagnostic Apparatus

Next, the operation of the ultrasonic diagnostic apparatus 11 will be briefly described. To transmit ultrasonic waves, a pulse signal is supplied to the piezoelectric elements 25. The pulse signal is supplied to the elements 23 on a row-by-row basis through the bottom electrode terminals 35 and 37 and the top electrode terminals 34 and 36. In each of the elements 23, an electric field acts on the piezoelectric film 28 between the bottom electrode 27 and the top electrode 26. The piezoelectric film 28 vibrates at the frequency of ultrasonic waves. The vibration of the piezoelectric film 28 is transferred to the vibration film 24. Thus, the vibration film 24 vibrates ultrasonically. As a result, a desired ultrasonic beam is emitted toward the subject (for example, the interior of a human body).

Reflected waves of the ultrasonic waves vibrate the vibration film 24. The ultrasonic vibration of the vibration film 24 ultrasonically vibrates the piezoelectric film 28 at a desired frequency. A voltage is output from the piezoelectric element 25 in accordance with the piezoelectric effect of the piezoelectric element 25. In each of the elements 23, a potential is generated between the top electrode 26 and the bottom electrode 27. The generated potentials are output from the bottom electrode terminals 35 and 37 and the top electrode terminals 34 and 36 as electric signals. The ultrasonic waves are detected in this manner.

Ultrasonic waves are repeatedly transmitted and received. As a result, a linear scan or a sector scan is achieved. When the scan is completed, an image is formed based on digital signals of the output signals. The image thus formed is displayed on the screen of the display panel 15.

The connecting body 65 formed of the second resin material is directly coupled to the thin portion 66 formed of the first resin material. Therefore, formation of an adhesive layer between the thin portion 66 and the connecting body 65 can be avoided. Even though the acoustic lens 18 is a coupled body of the first resin material and the second resin material, the acoustic lens 18 can be free from the influence of an adhesive. The acoustic lens 18 that is not affected by the film thickness of an adhesive is thus provided.

The second resin material has a Shore hardness value that is smaller than that of the first resin material. The partial cylindrical surface 18a of the acoustic lens 18 comes into contact with an object. Ultrasonic waves propagate through the thin portion 66 and the connecting body 65. Since the thin portion 66 has a Shore hardness value that is larger than that of the connecting body 65, the thin portion 66 can exhibit higher resistance to wear than the connecting body 65.

The second resin material contains filler, the value of the amount of filler that is contained being smaller than that of the first resin material. The acoustic impedance of the second resin material can be reduced according to the amount of filler that is reduced. Therefore, the overall acoustic impedance of the acoustic lens 18 is reduced when compared with the case where the acoustic lens 18 is entirely formed of the first resin material. Ultrasonic waves propagating through the acoustic lens 18 are thus increased. The sensitivity with respect to ultrasonic waves can be enhanced.

The thick portion 67 is formed in a frame shape that surrounds the perimeter of the thin portion 66. The thick portion 67 is less likely to be broken than the thin portion 66. Since the edge of the thin portion 66 is continuously surrounded by the thick portion 67, breakage of the thin portion 66 can be sufficiently avoided during, for example, mold release.

(4) Method for Manufacturing Acoustic Lens

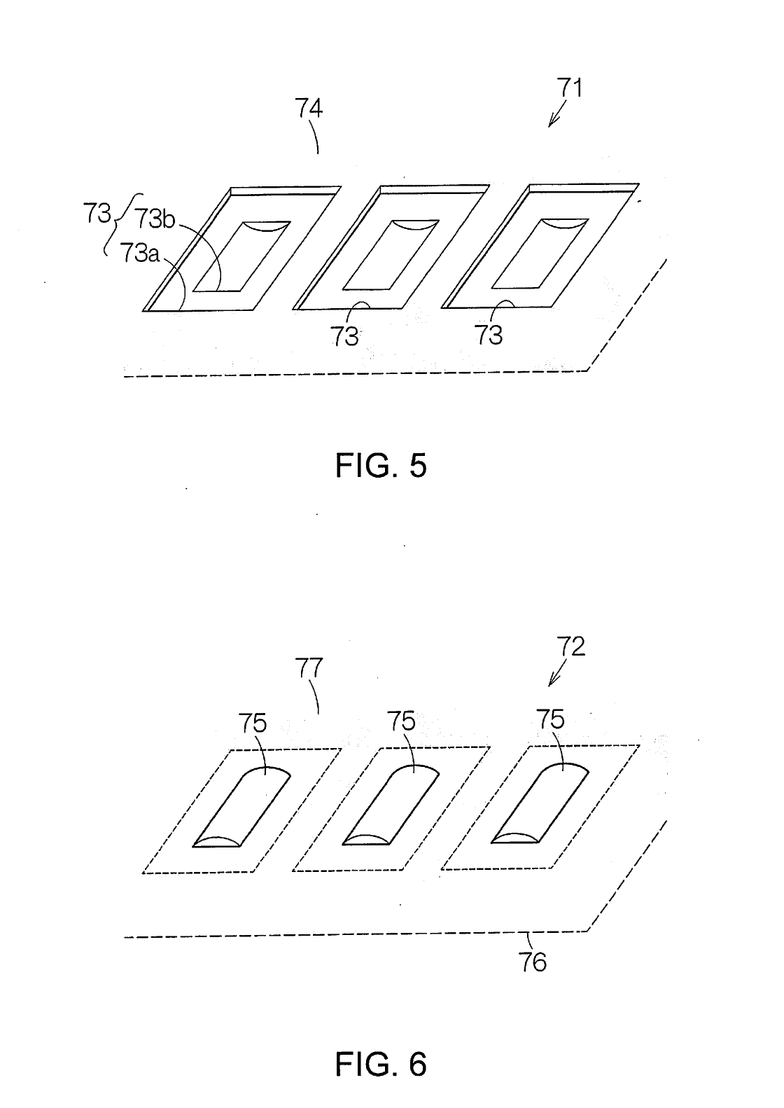

Next, a method for manufacturing the acoustic lens 18 will be described in detail. Compression molding is used to manufacture the acoustic lens 18. A first mold 71 and a second mold 72 are prepared. The first mold 71 and the second mold 72 can be formed from a metal material. As shown in FIG. 5, spaces 73 that each define the shape of the acoustic lens 18 are demarcated in the first mold 71. The spaces 73 are recessed from a plane 74. The reference plane RF of the acoustic lens 18 matches the plane 74 of the first mold 71. Each space 73 is constituted by a flat plate space 73a that is recessed from the plane 74 and that defines the shape of the flat plate portion 18b of the acoustic lens 18, and a depression 73b that is further recessed from the flat plate space 73a and that defines the shape of the partial cylindrical surface 18a. The depression 73b is demarcated by a partial cylindrical surface having generating lines that are parallel to the plane 74. In the first mold 71, only one space 73 may be demarcated, or a plurality of spaces 73 may be arranged as shown in FIG. 5. The first resin material in fluid form is supplied into the spaces 73.

As shown in FIG. 6, the second mold 72 has protruding surface portions 75 that respectively correspond to the spaces 73 of the first mold 71. The protruding surface portions 75 protrude from a reference plane 76 of the second mold 72 by a certain height. Each protruding surface portion 75 is demarcated by a partial cylindrical surface having generating lines that are parallel to the reference plane 76. When the second mold 72 is laid on top of the first mold 71, the generating lines of the protruding surface portion 75 are located parallel to the generating lines of the corresponding depression 73b. The protruding surface portion 75 is positioned facing a curved surface of the depression 73b.

A flat surface portion 77 that extends in the reference plane 76 continuously with the protruding surface portions 75 is defined in the second mold 72. The flat surface portion 77 has regions corresponding to the respective outlines of the spaces 73 of the first mold 71. When the second mold 72 is laid on top of the first mold 71, the outer edges of those regions of the flat surface portion 77 coincide with the outer edges of the corresponding spaces 73.

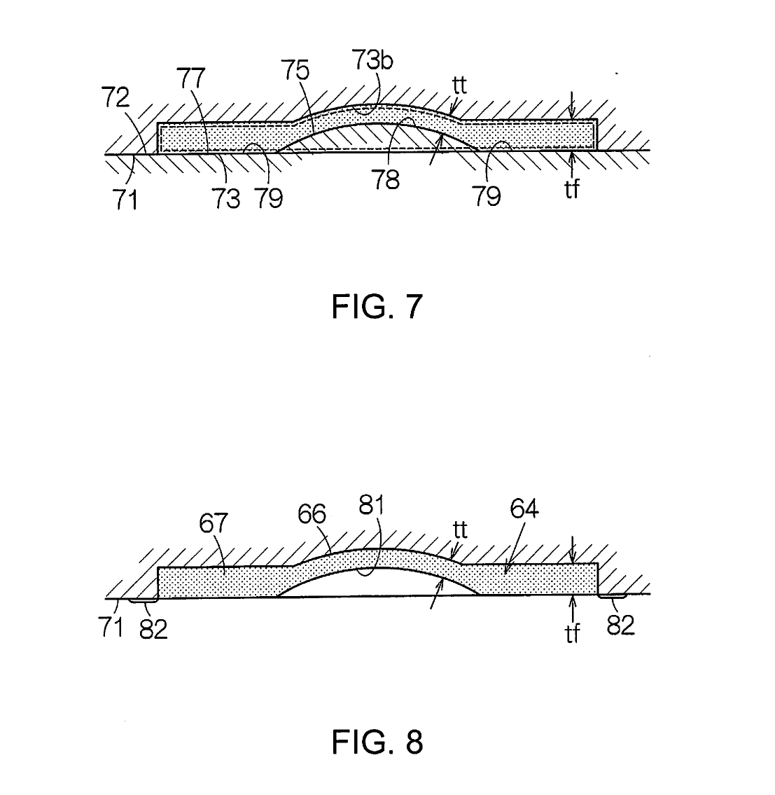

As shown in FIG. 7, when the second mold 72 is laid on top of the first mold 71, the spaces 73 of the first mold 71 are closed by the flat surface portion 77 of the second mold 72. The spaces 73 are hermetically sealed. The closed spaces 73 are filled with the first resin material in fluid form. The protruding surface portions 75 of the second mold 72 enter the respective spaces 73 of the first mold 71. Each protruding surface portion 75 is positioned facing the curved surface of the corresponding depression 73b. A first cavity 78 filled with the first resin material is formed between the curved surface of the depression 73b and the protruding surface portion 75. Similarly, a second cavity 79 filled with the first resin material is formed between the first mold 71 and the flat surface portion 77 around the depression 73b. The second cavity 79 is continuous with the first cavity 78. The cavity thickness tf of the second cavity 79 is larger than the cavity thickness tt of the first cavity 78.

After that, the first resin material is hardened. As shown in FIG. 8, the outer covering (resin body) 64 is thus molded. The outer covering 64 has the thin portion 66 located within the first cavity 78 and the thick portion 67 located within the second cavity 79. The thick portion 67 extends outward from the outline of the thin portion 66 continuously therewith. The thickness tf of the thick portion 67 is larger than the thickness tt of the thin portion 66. After the first resin material is cured, the second mold 72 is detached from the first mold 71. The protruding surface portion 75 serves to form a depression 81 in the outer covering 64. The curved surface of the depression 81 reflects the partial cylindrical surface of the protruding surface portion 75. At this time, the first resin material overflows from the space 73 to a gap, around the space 73, between the first mold 71 and the second mold 72. The overflowing first resin material forms flash 82.

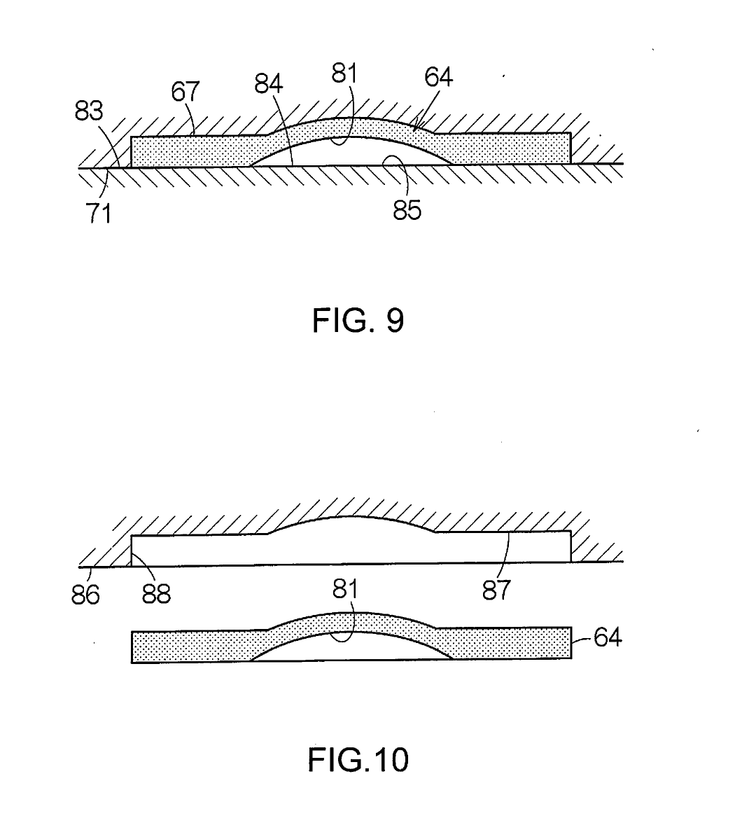

As shown in FIG. 9, a third mold 83 is laid on top of the first mold 71. Before the third mold 83 is laid on top of the first mold 71, the second resin material in fluid form is supplied into the depression 81 of the outer covering 64. A flat inner wall surface 84 of the third mold 83 is in contact with the thick portion 67 of the outer covering 64. When the third mold 83 is laid on top of the first mold 71, the depression 81 of the outer covering 64 is closed by the inner wall surface 84. The depression 81 is hermetically sealed. A space 85 that is thus closed is filled with the second resin material. The space 85 filled with the second resin material is formed between the depression 81 of the outer covering 64 and the inner wall surface 84 of the third mold 83.

After that, the second resin material is hardened. The second resin material is hardened such that it is continuous with the outer covering 64. The connecting body 65 is thus formed. In this manner, the acoustic lens 18 is molded. The third mold 83 is detached from the first mold 71. Subsequently, the acoustic lens 18 is released from the first mold 71. During mold release, a worker can hold the flash 82. Accordingly, the acoustic lens 18 can be easily pulled out of the first mold 71. After the mold release, the flash 82 is removed.

The second resin material is cured into a shape that conforms to the depression 81 of the outer covering 64. At the same time as the hardening of the second resin material, the cured product of the second resin material is coupled to the outer covering 64 of the first resin material. Therefore, formation of an adhesive layer between the outer covering 64 and the connecting body 65 can be avoided. Thus, even though the acoustic lens 18 is a coupled body of the outer covering 64 and the connecting body 65, the acoustic lens 18 can be free from the influence of an adhesive. The method for manufacturing the acoustic lens 18 that is not affected by the film thickness of an adhesive is thus provided.

In this manufacturing method, after curing of the first resin material, the second mold 72 is detached from the first mold 71, and the third mold 83 is laid on top of the first mold 71 with the outer covering 64 remaining on the first mold 71. The molded outer covering 64 is held on the first mold 71 without being released from the first mold 71. During curing of the second resin material, the first mold 71 is used as is, so that mold-use efficiency is increased. The manufacturing cost is thus suppressed.

Here, the thick portion 67 of the outer covering 64 is formed into a frame shape that surrounds the perimeter of the thin portion 66. The thick portion 67 is less likely to be broken than the thin portion 66. Since the edge of the thin portion 66 is continuously surrounded by the thick portion 67, breakage of the thin portion 66 can be effectively avoided during, for example, mold release.

In this manufacturing method, it is also possible that after curing of the first resin material, the outer covering 64 is temporarily released from the first mold 71. In this case, as shown in FIG. 10, for example, a fourth mold 86 can be used to mold the connecting body 65 formed of the second resin material. The fourth mold 86 has a cavity surface 87 having the same shape as the first mold 71. The cavity surface 87 of the fourth mold 86 demarcates a space having the same shape as the spaces 73 of the first mold 71. The outer covering 64 is transferred onto the fourth mold 86. After that, the second resin material is supplied into the depression 81 of the outer covering 64. After the second resin material is supplied, the third mold 83 is laid on top of the fourth mold 86. In this method, the molded outer covering 64 is temporarily released from the first mold 71. The outer covering 64 is transferred from the first mold 71 onto the fourth mold 86. After the transfer, the third mold 83 is laid on top of the fourth mold 86. The time and effort taken to transport the first mold 71 can be eliminated.

(5) Acoustic Lens According to Another Embodiment

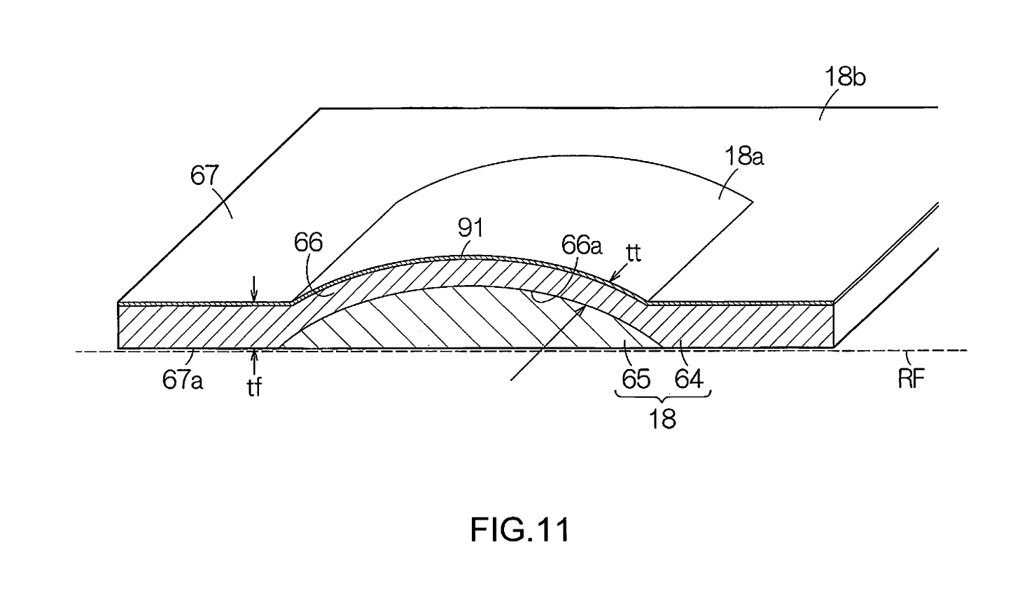

As shown in FIG. 11, during formation of the partial cylindrical surface 18a, a coating film of a third resin material may be formed on the outer surface of the outer covering 64. The coating film can be formed of, for example, an urethane coating layer. The urethane coating layer protects the surface of the acoustic lens 18 against wear. To form this coating film, the third resin material can be applied to the surface of the outer covering 64 that conforms to the first mold 71. The third resin material can be hardened such that it has a uniform film thickness.

(6) Acoustic Coupling Member that May Substitute for Acoustic Lens

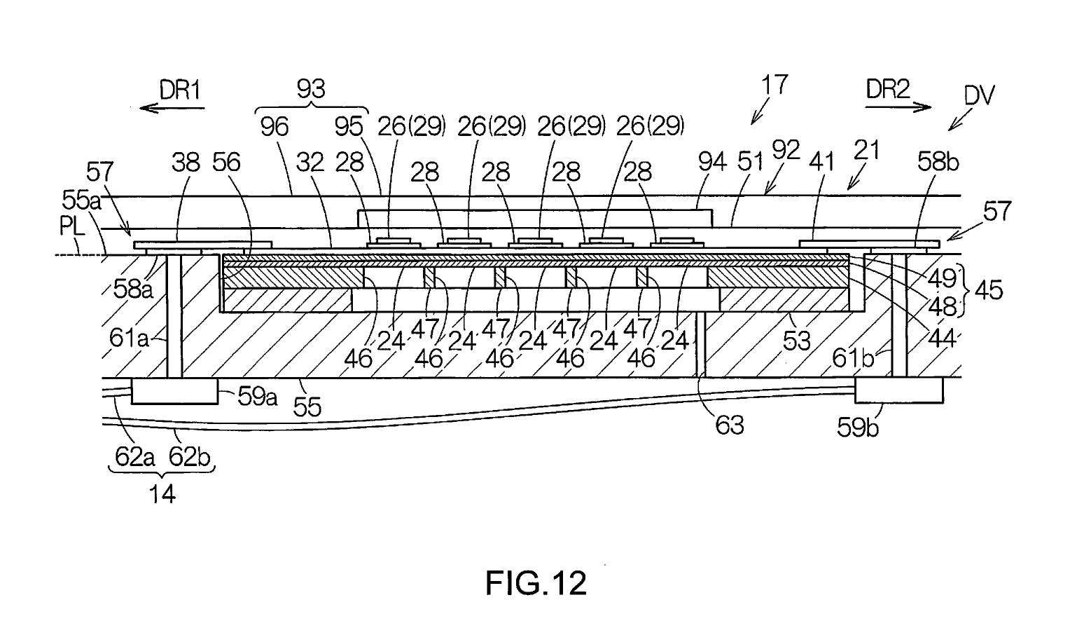

As shown in FIG. 12, to cause an ultrasonic beam to converge, a scan operation of the elements 23 may be used instead of the acoustic lens 18. In this case, instead of the second electric conductors 32 that are connected commonly to a plurality of elements 23 in the row direction of the arrangement, a signal line is formed for each element 23. An acoustic coupling member 92 is coupled to the element array 22 via the acoustic matching layer 52. The acoustic coupling member 92 is formed to have a two-layer structure. The acoustic coupling member 92 includes an outer covering 93 and a connecting body (connecting portion) 94. The outer covering 93 is formed of the first resin material in the same manner as described above. The connecting body 94 is formed of the second resin material in the same manner as described above. The outer covering 93 has a thin portion 95 and a thick portion 96. The thin portion 95 is shaped into a flat plate shape. Similarly, the connecting body 94 is shaped into a flat plate shape. The thin portion 95 is laid on top of the connecting body 94. The connecting body 94 is in close contact with the thin portion 95. The other structures are the same as those of the above-described ultrasonic device 17.

Although some embodiments of the invention have been described in detail above, a person skilled in the art will readily understand that various modifications may be made without substantially departing from the novel teachings and the effects of the invention. Therefore, such modifications are entirely included within the scope of the invention. For example, any term described at least once together with a broader or synonymous different term in the specification or the drawings may be replaced by the different term at any place in the specification or the drawings. Moreover, the configurations and operations of the ultrasonic diagnostic apparatus 11, the device terminal 12, the ultrasonic probe 13, the display panel 15, the housing 16, the base 21, the elements 23, the first and second wiring boards 38 and 41, the acoustic matching layer 51, and the like are not limited to those described in the foregoing embodiments, but may be modified in various manners.

The entire disclosure of Japanese Patent Application No. 2014-156709 filed on Jul. 31, 2014 is expressly incorporated by reference herein.

What is claimed is:

1. A method for manufacturing an acoustic coupling member, the method comprising:
laying, on top of a first mold that has a recessed surface portion, a second mold that has a protruding surface portion having a certain height from a reference plane and a flat surface portion extending in the reference plane continuously with the protruding surface portion, thereby forming a first cavity between the recessed surface portion and the protruding surface portion and a second cavity between the first mold and the flat surface portion, where the second cavity is continuous with the first cavity and has a larger cavity thickness than a cavity thickness of the first cavity, and filling the first cavity and the second cavity with a first resin material;
hardening the first resin material, thereby molding a resin body having a thin portion located within the first cavity and a thick portion located within the second cavity, the thick portion extending outward from an outline of the thin portion continuously therewith and having a larger thickness than a thickness of the thin portion;
laying a third mold, the third mold having a flat inner wall surface that comes into contact with the thick portion of the resin body, on top of the first mold, thereby forming a space between the inner wall surface and a depression that is formed in the resin body so as to correspond to the protruding surface portion, and filling the space with a second resin material; and
hardening the second resin material such that the second resin material is continuous with the resin body, thereby molding an acoustic coupling member.

2. The method according to claim 1,
wherein after the first resin material is cured, the second mold is detached from the first mold, and the third mold is laid on top of the first mold with the resin body remaining on the first mold.

3. The method according to claim 1,
wherein after the first resin material is cured, the resin body is released from the first mold, and the resin body is transferred onto a mold having a cavity surface that has the same shape as the first mold.

4. The method according to claim 1,
wherein flash is formed between the first mold and the second mold, extending outward from an outline of the resin body.

5. The method according to claim 1, further comprising:
applying a third resin material to a surface of the resin body, the surface conforming to the first mold.

6. The method according to claim 1,
wherein the protruding surface portion constitutes a partial cylindrical surface having generating lines that are parallel to the reference plane, and the protruding surface portion is positioned facing the recessed surface portion that is defined in the first mold, the recessed surface portion constituting a depression that constitutes a partial cylindrical surface having generating lines that are parallel to said generating lines, and demarcates the first cavity between the protruding surface portion and the first mold.

7. The method according to claim 1,
wherein the thick portion is formed into a frame shape that surrounds a perimeter of the thin portion.

8. An acoustic coupling member, comprising:
a thin portion that is formed of a first resin material and that extends, at a position separated from a reference plane, along the reference plane;
a thick portion that is formed of the first resin material, that has a larger thickness than a thickness of the thin portion, and that defines a flat surface, the flat surface extending outward from an outline of the thin portion continuously therewith and being in contact with the reference plane; and
a connecting portion that is formed of a second resin material, the second resin material being different from the first resin material, and that fills a space between the reference plane and the thin portion and is in close contact with the thin portion.

9. The acoustic coupling member according to claim 8,
wherein the second resin material has a Shore hardness value that is smaller than that of the first resin material.

10. The acoustic coupling member according to claim 8,
wherein the second resin material contains filler, a value of an amount of said filler that is contained being smaller than that of the first resin material.

11. The acoustic coupling member according to claim 8,
wherein the thick portion is formed into a frame shape that surrounds a perimeter of the thin portion.

12. The acoustic coupling member according to claim 8,
wherein a surface of the thin portion, the surface being located on a side opposite to the reference plane, is formed by a partial cylindrical surface having generating lines that are parallel to the reference plane.

13. A probe, comprising:
the acoustic coupling member according to claim 8;
an ultrasonic device that is coupled to the acoustic coupling member; and
a housing that supports the ultrasonic device.

14. An electronic apparatus, comprising:
the acoustic coupling member according to claim 8;
an ultrasonic device that is coupled to the acoustic coupling member; and
a processing unit that is connected to the ultrasonic device and processes an output from the ultrasonic device.

15. An ultrasonic imaging apparatus, comprising:
the acoustic coupling member according to claim 8;
an ultrasonic device that is coupled to the acoustic coupling member;
a processing unit that is connected to the ultrasonic device, processes an output from the ultrasonic device, and generates an image; and
a display device that displays the image.

16. An acoustic coupling member, comprising:
a first acoustic coupling layer that is provided on a reference plane; and
a second acoustic coupling layer that is provided on the reference plane and that covers the first acoustic coupling layer,
wherein a hardness of the first acoustic coupling layer is smaller than a hardness of the second acoustic coupling layer.

17. The acoustic coupling member according to claim 16,
wherein a thickness of a region of the second acoustic coupling layer that is in contact with the reference plane is larger than a thickness of a region of the second acoustic coupling layer that is in contact with the first acoustic coupling layer.

18. An ultrasonic probe, comprising:
an ultrasonic device;
a first acoustic coupling layer that is provided on the ultrasonic device; and
a second acoustic coupling layer that is provided on the ultrasonic device and that covers the first acoustic coupling layer,
wherein a hardness of the first acoustic coupling layer is smaller than a hardness of the second acoustic coupling layer.

19. The ultrasonic probe according to claim 18,
wherein a thickness of a region of the second acoustic coupling layer that is in contact with the ultrasonic device is larger than a region of the second acoustic coupling layer that is in contact with the first acoustic coupling layer.
